(19) United States
(12) Patent Application Publication
(10) Pub. No.: US 2023/0093261 A1
(43) Pub. Date: Mar. 23, 2023

(54) Oral Care Substance Delivery Device and Method of Manufacture

(71) Applicants: OraLine, Inc., Cortland, NY (US); Lucky Red Chicken, Inc., Newfield, NY (US)

(72) Inventors: Andrew Turco, Ithaca, NY (US); Wendy Ives, Newfield, NY (US)

(21) Appl. No.: 17/482,721

(22) Filed: Sep. 23, 2021

(51) Int. Cl. A61K 8/98; A61C 19/06; A61K 8/02; A61K 8/9789; A61K 8/34; A61K 8/19; A61Q 11/00

(52) U.S. Cl. CPC A61K 8/988; A61C 19/063; A61K 8/0216; A61K 8/9789; A61K 8/345; A61K 8/19; A61Q 11/00

(57) ABSTRACT

A device to deliver an oral care substance to a tooth surface or a gingival tissue surface includes a body and an oral care substance. The body is orally acceptable and malleable, including beeswax and having a first surface configured to be applied to the tooth surface and/or the adjoining gingival tissue surface. The first surface is configured to adhere to the tooth surface and/or the adjoining gingival tissue surface. The oral care substance is deposited on the first surface and/or impregnated into the body, the substance being configured to contact the tooth surface and/or the adjoining gingival tissue surface.

### BACKGROUND OF THE INVENTION

### Field of the Invention

The invention pertains to the field of oral care substance delivery devices, and more particularly, to strips to deliver oral care substances to teeth and gingival tissues.

### Description of Related Art

Delivery devices are known for delivering an oral healthcare substance to the surface of a tooth and other oral tissue. Such delivery devices include a thin strip of flexible, flimsy film coated with an oral healthcare substance, such as gel containing fluoride and hydrogen peroxide. A coated side of the flexible film is mated to a backing material, such as a plastic sheet with more rigidity than the flimsy film. The film and backing material are packaged in a coated aluminum foil wrapping. To use the delivery device, the wrapping is opened to reveal the gel-covered film, which is peeled or slid from the backing material and then applied with the gel-coated side against the surface of the tooth or other oral tissue.

These delivery devices are difficult and unpleasant to handle. The gel is messy when removing the film and backing material from the wrapping, and when peeling the film from the backing material, and the thin overly flexible film can slide around on the backing material, fold over on itself during handling, and generally be difficult to apply properly and cleanly to a desired portion of the mouth.

### SUMMARY OF THE INVENTION

An oral care substance delivery device is disclosed herein that is more effective and comfortable to use, neater and easier to handle, and less expensive to manufacture than conventional oral care substance delivery devices. A method to manufacture the device is also disclosed.

In an embodiment, a device to deliver an oral care substance to a tooth surface or a gingival tissue surface includes: a body, the body being orally acceptable and malleable, the body having a first surface configured to be applied to the tooth surface and/or the adjoining gingival tissue surface, the first surface configured to adhere to the tooth surface and/or the adjoining gingival tissue surface, the body including beeswax; and an oral care substance deposited on the first surface and/or impregnated into the body, the substance configured to contact the tooth surface and/or the adjoining gingival tissue surface.

In another embodiment, a method of making a device to deliver an oral care substance to a tooth surface or a gingival tissue surface, includes: heating solid beeswax to at least the melting point of the beeswax to yield liquid beeswax; pouring the liquid beeswax through rollers, the rollers configured to output a beeswax sheet; and cutting cut the beeswax sheet into portions of a predetermined size.

### BRIEF DESCRIPTION OF THE DRAWING

FIG. 1 shows a perspective view of an oral care substance delivery device, according to an embodiment.

FIG. 2 shows a perspective view of an oral care substance delivery device, according to an embodiment.

FIG. 3 shows a perspective view of the oral care substance delivery device of FIG. 1, with a wrapper partly cut away, according to an embodiment.

FIG. 4 shows a step of a method of manufacturing an oral care substance delivery device.

FIG. 5 shows a step of a method of manufacturing an oral care substance delivery device.

FIG. 6 shows a step of a method of manufacturing an oral care substance delivery device.

FIG. 7 shows a step of a method of manufacturing an oral care substance delivery device.

### DETAILED DESCRIPTION OF THE INVENTION

In the following description, reference is made to the accompanying drawings that form a part thereof, and in which is shown by way of illustration specific example embodiments in which the present teachings may be practiced. These embodiments are described in sufficient detail to enable those skilled in the art to practice the present teachings and it is to be understood that other embodiments may be utilized and that changes may be made without departing from the scope of the present teachings. The following description is, therefore, merely exemplary.

The terminology used herein is for the purpose of describing particular example embodiments only and is not intended to be limiting. As used herein, the singular forms “a”, “an”, and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The terms “approximately” and “about”, when qualifying a quantity, shall mean the quantity with a tolerance plus or minus 10 percent of the quantity, unless otherwise specified.

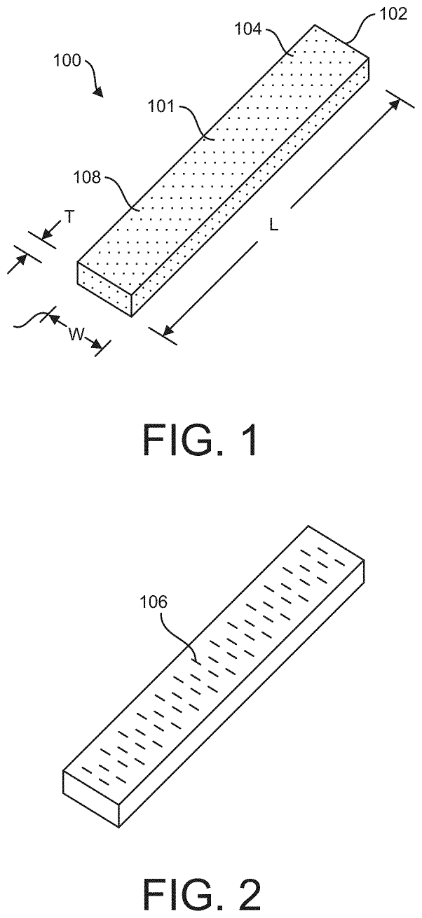

FIG. 1 shows a perspective view of an oral care substance delivery device 100 intended to deliver an oral care substance or additive 101 to the teeth and/or gingival tissue. Referring to FIG. 1, the oral care substance delivery device 100 includes an orally acceptable, malleable body or portion 102. The portion 102 shown in the figures is shaped as a strip or rectangle, though the portion 102 can be shaped variously as desired to fit onto a desired oral area. The portion 102 has a first surface 104 configured to be applied to a tooth surface and/or adjoining gingival tissue surface. It should be understood that “a tooth surface” includes one tooth surface or the surfaces of a row of teeth. The first surface 104 is configured to adhere to the tooth surface and/or the adjoining gingival tissue surface. The first surface 104 can have a surface texturing 106, an example of which is shown in FIG. 2, to facilitate adherence to the tooth surface. In some embodiments, the portion 102 has a length L about 50-75 mm long, which is long enough, for example, to coat multiple teeth. In some embodiments, the width W is about 10-15 mm wide, which is wide enough to coat a range of tooth sizes from the gum to a tooth end opposite the gum. In some cases, the width W is large enough that the portion 102 can overlap and coat at least a portion of the gum, and in some cases the width W is large enough that the portion 102 can be bent around the tooth end to cover the tooth end, and even to coat at least a portion of a back tooth surface. In some embodiments, the portion 102 can be about 0.2-3.0 mm thick. In this thickness range, the portion 102 is sufficiently malleable to be bent and conformed to the tooth surface, to the contours around the edges of the tooth surface, and/or into the gap between two directly adjacent teeth.

The portion 102 comprises primarily beeswax 108. In some embodiments, beeswax 108 accounts for 50% to 97%, by weight, of the composition of the portion 102. In some embodiments, the beeswax can account for 90% or more, by weight, of the composition of the portion 102. The portion 102 can also include a variety of substances or additives 101 beneficial for oral care (e.g., tooth whiteners, anti-microbial agents, anti-inflammatory agents), processing, or use (e.g., flavorings), such as hydrogen peroxide, fluoride, papain, coconut oil, olive oil, tea tree oil, propolis, bee pollen, activated charcoal, sea salt, lemon oil, orange zest, peppermint oil, spearmint oil, sage oil, clove oil, xylitol, stevia, banana powder, plantain extract, apple powder, and strawberry powder. The additives 101 can be all natural. For example, natural substances papain, sea salt, lemon oil, sage oil, and coconut oil, facilitate whitening. Since beeswax is natural and the additives 101 can be all natural, the oral care substance delivery device 100 can be all natural.

The additives 101 can be applied to coat the beeswax 108 or blended homogenously with the beeswax 108. The portion 102 can be colored by the additives 101, or colored to correspond with the additives 101, to be consistent with a color expectation a consumer might have for an additive. Colors, for example, can be white, orange, or black. The portion 102 can also be transparent or translucent to achieve less noticeability when worn in a consumer's mouth.

Beeswax has several important benefits in the oral care substance delivery device 100. For example, beeswax has intrinsic qualities beneficial to oral care, such as anti-microbial properties resulting from, for example, residues of propolis and bee pollen in the beeswax.

Further, solid beeswax has a certain degree of rigidity such that a consumer can easily handle the portion 102. Without any manual force or other external force besides the force of Earth's gravity, and without any external support applied to the portion 102, the portion 102 retains its general shape (e.g., within 10% of its shape, excluding some degree of viscous flow that might occur over a relatively long time (e.g., months, years, or longer). Simultaneously, beeswax has a certain degree of malleability and moldability such that with the application of relatively light external force, a consumer can easily bend and press the portion 102 to conform against the tooth surface and/or into the gap between two directly adjacent teeth. The conformance of the portion 102 facilitates adherence of the portion 102 to the tooth surface. When the portion 102 is conformed to multiple teeth, the portion 102 can be pressed into the space between individual teeth to further facilitate adherence. Similarly, the portion 102 can be wrapped around the bottom of one or more teeth to further facilitate adherence. The portion 102, with its degree of rigidity, retains the molded shape adhered to the tooth surface unless another external force acts to reshape the portion 102.

The beeswax 108 can also be anhydrous, and the portion 102 can have little or no additional moisture added, including from the additives 101 in the portion 102, which can be applied to the first surface 104 of the portion 102 in some embodiments, and can be impregnated in the portion 102 or mixed with the beeswax 108 in some embodiments. In some embodiments, the beeswax 108 can have less than 5% moisture content. In other embodiments, the beeswax 108 can have less than 1% moisture content. In other embodiments, the beeswax 108 can have less than 0.1% or 0% moisture content. In some embodiments, the additives 101 can add some moisture content to the oral care substance delivery device 100. In these embodiments, in some instances, the moisture content of the oral care substance delivery device 100 can be less than 25%. In some instances, the moisture content of the oral care substance delivery device 100 can be less than 15%. In some instances, the moisture content of the oral care substance delivery device 100 can be less than 10%, less than 5%, less than 1%, or less than 0.1%. In some embodiments, the beeswax 108 and the additives can add 0% moisture to the oral care substance delivery device 100.

In each case, the texture and consistency of the portion 102 is determined dominantly by the beeswax 108, such that the portion 102 can have a waxy texture or consistency dissimilar to the gel coating of conventional art whitening strips. Having a dry, approximately dry, or relatively dry surface lacking the gel of conventional art whitening strips facilitates easier handling by consumers. Use of the oral care substance delivery device 100 is also less messy in the mouth.

In embodiments of the portion 102 with the additives 101 impregnated or mixed into the beeswax 108, the beeswax 108 can encapsulate the additives 101 and release the additives onto the tooth surface and gingival tissue during use. The beeswax can act as a humectant, drawing moisture from the tooth or gingival tissue, and the beeswax 108 can have a permeable and crystalline structure, all of which facilitates release of the additives from the beeswax. Additives can be released from the portion 102 over an amount of time. This release of additives from the beeswax over time avoids the mess and uncomfortable feel of gel accidentally applied, or inadvertently spilling, onto undesired areas of the mouth. A consumer can wear the oral care substance delivery device for a relatively long time, or an indefinite amount of time, to allow a desired amount of release of additives 101, without harmful side effects because the beeswax 108 and the additives 101 can be all natural, and/or included in amounts or concentrations known to cause no harmful side effects.

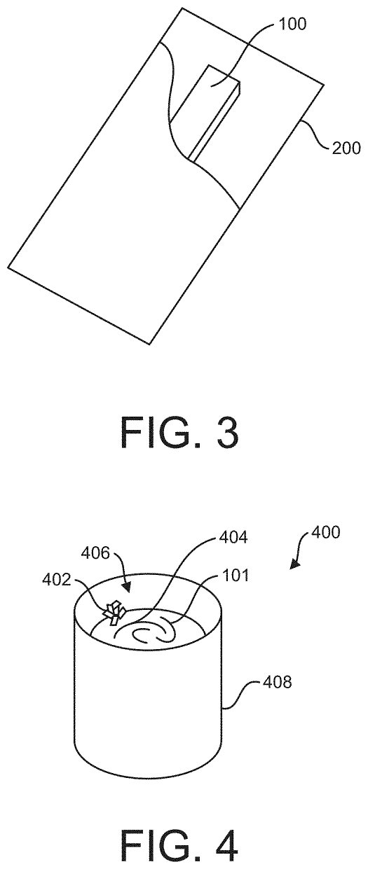

FIG. 3 shows a perspective view of the oral care substance delivery device 100 in packaging 200 partly cut away. The packaging 200 can be a pouch made of paper, coated paper, plastic, or another known packaging material, in which the portion 102, including the additives 101 and the beeswax 108, are covered, protected, and/or preserved. Unwrapping the packaging 200 to use the oral care substance delivery device 100 is significantly cleaner and easier than with conventional devices containing gel because the oral care substance delivery device 100 is dry or approximately dry and the portion 102 is placed directly inside the packaging 200 with no backing material necessary for support. The wrapping can simply be torn or cut open, and the portion 102 can easily be handled to place and press the portion 102 on the desired oral location.

FIGS. 4-7 illustrate a method of manufacturing an oral care substance delivery device 100. Step 400, illustrated in FIG. 4, includes heating solid beeswax 402 to at least the melting point of the beeswax to yield liquid beeswax 404. In some embodiments, the beeswax 402, 404 can be heated to 65-70 degrees Celsius. The solid beeswax 402 can be solid portions, such as pellets or blocks. Smaller portions can be heated more quickly. The additives 101 can be added before or during step 400 to create a liquid beeswax mixture 406. The beeswax 402, 404 can be heated by any appropriate, now-known or future-developed heating method. In the embodiment illustrated in FIG. 4, the solid beeswax 402 is melted in a heated tank 408.

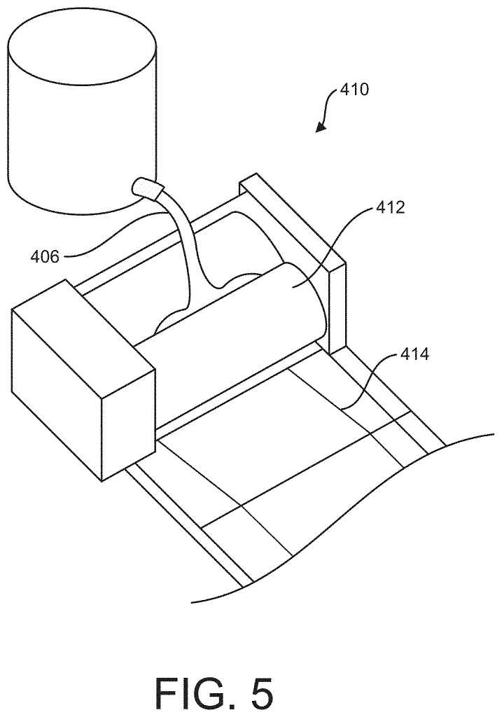

Step 410, illustrated in FIG. 5, includes pouring the liquid beeswax mixture 406 through first rollers 412, which compress the beeswax as it cools to output a beeswax sheet 414. The beeswax crystallizes quickly as it cools, which can happen abruptly as the beeswax hits the solid surface of the first rollers 412 and can instantly cause the beeswax to crystallize. The crystals forming in the cooling liquid beeswax mixture 406 and the beeswax sheet 414 encapsulate the additives 101 and/or the naturally occurring ingredients of beeswax (e.g., propolis and bee pollen).

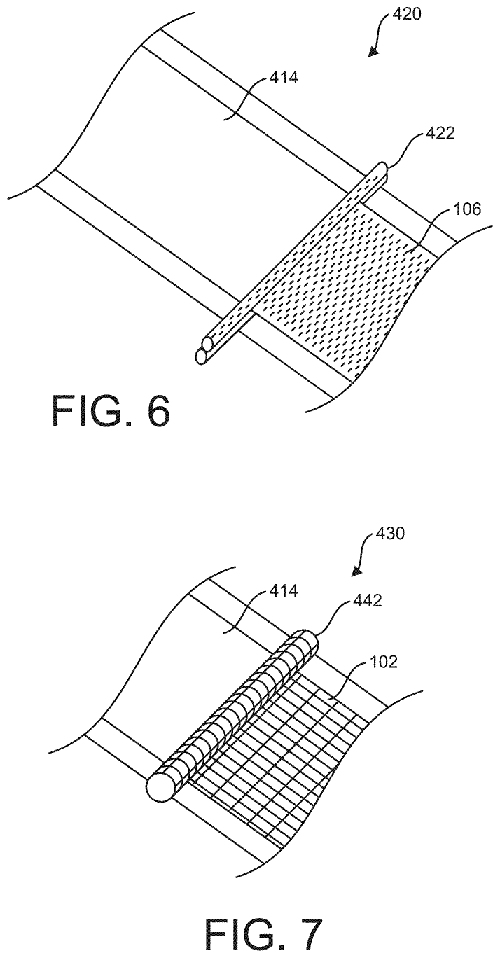

Step 420, illustrated in FIG. 6, includes compressing the beeswax sheet 414 by passing the beeswax sheet 414 through second rollers 422. The second rollers 422 can be heated to increase the viscosity and malleability of the beeswax sheet 414. This step 420 can be repeated as desired to compress the beeswax sheet 414 to a desired thickness. Compressing the beeswax sheet 414 stretches the beeswax sheet 414 and draws the beneficial macromolecules of the additives 101 closer to the surfaces of the beeswax sheet 414, including what will become the first surface 104.

The first rollers 412 in step 410 and/or the second rollers 422 in step 420 can have a patterned surface, as illustrated in FIG. 6, to impart the surface texturing 106 (see also FIG. 2) on the surface of the beeswax sheet 414. The texture on the first rollers 412 and/or the second rollers 422 can also help move the beeswax sheet 414 consistently through the first rollers 412 and/or the second rollers 422.

The beeswax sheet 414 can form a crystalline structure to encapsulate the additives 101 and/or the naturally occurring ingredients of beeswax (e.g., propolis and bee pollen) during cooling in step 420. Additives 101 can also be added directly to one or more surfaces of the beeswax sheet 414. The additives can be added in a solution form to permeate a depth into the beeswax sheet 414, after which the solution can dry, leaving dry additive 101 impregnating the beeswax sheet 414.

When the beeswax sheet 414 has been compressed to a desired thickness T (see FIG. 1), and has sufficiently cooled, according to step 430, as illustrated in FIG. 7, the beeswax sheet 414 is cut by a cutter 442 to desirably sized portions 102 of predetermined length L and width W (see FIG. 1). Each portion 102 can be wrapped to preserve the oral care substance delivery device 100 and keep the oral care substance delivery device 100 clean until a consumer is ready to use the oral care substance delivery device 100. During use, the consumer can wet the first surface 104 of the oral care substance delivery device 100 intended to be applied to the teeth and/or gingival tissue, in order to “activate” or facilitate release of the additive(s) from the oral care substance delivery device.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A device to deliver an oral care substance to a tooth surface or a gingival tissue surface, the device comprising:
a body, the body being orally acceptable and malleable, the body having a first surface configured to be applied to the tooth surface and/or the adjoining gingival tissue surface, the first surface configured to adhere to the tooth surface and/or the adjoining gingival tissue surface, the body including beeswax; and
an oral care substance deposited on the first surface and/or impregnated into the body, the substance configured to contact the tooth surface and/or the adjoining gingival tissue surface.

2. The device of claim 1, wherein the beeswax is anhydrous.

3. The device of claim 1, wherein the first surface of the body is dry.

4. The device of claim 1, wherein the body has a moisture content less than 15%

5. The device of claim 1, wherein the body has a shape of a strip.

6. The device of claim 1, wherein a majority of the body is beeswax.

7. The device of claim 1, wherein 50% to 97% by weight is beeswax.

8. The device of claim 1, wherein the body is at least 90% beeswax.

9. The device of claim 1, wherein the body further comprises one or more of papain, coconut oil, olive oil, tea tree oil, propolis, bee pollen, activated charcoal, sea salt, lemon oil, orange zest, peppermint oil, spearmint oil, sage oil, clove oil, xylitol, stevia, banana powder, plantain extract, apple powder, and strawberry powder.

10. The device of claim 1, wherein the body includes at least one color from the group consisting of transparent, translucent, white, orange, and black.

11. The device of claim 1, further comprising packaging enclosing the body.

12. The device of claim 11, wherein the body is supported directly and only by the packaging.

13. The device of claim 1, wherein the body retains a general shape when unsupported externally and the only force applied to the body is Earth's gravitational force.

14. The device of claim 1, wherein the body has a length 50-75 mm, a width 10-15 mm, and a thickness 0.2-3.0 mm.

15. A method of making a device to deliver an oral care substance to a tooth surface or a gingival tissue surface, the method including:
heating solid beeswax to at least the melting point of the beeswax to yield liquid beeswax;
pouring the liquid beeswax through rollers, the rollers configured to output a beeswax sheet; and
cutting cut the beeswax sheet into portions of a predetermined size.

16. The method of claim 15, further comprising applying an oral care substance on a first surface of at least one of the beeswax sheet and the portions.

17. The method of claim 15, further comprising combining an oral care substance with at least one of the solid beeswax and the liquid beeswax.

18. The method of claim 15, further comprising impregnating at least one of the beeswax sheet and the portions with an oral care substance.

19. The method of claim 15, wherein at least one of the rollers has a textured or patterned exterior surface to impart a texture or pattern on the beeswax sheet.

20. The method of claim 15, further comprising passing the beeswax sheet through rollers to compress the beeswax sheet and reduce a thickness of the beeswax sheet.
